(19) United States
(12) Patent Application Publication
(10) Pub. No.: US 2004/0002520 A1
(43) Pub. Date: Jan. 1, 2004

(54) Composition and method for cessation of Nicotine cravings

(76) Inventors: Patrick L. Soderlund, New Prague, MN (US); Raymond E. Faulk, Mullins, SC (US)

Correspondence Address: Sean D. Solberg, DORSEY & WHITNEY LLP, Suite 1500, 50 South Sixth Street, Minneapolis, MN, 55402-1498, US

(21) Appl. No.: 10/395,520

(22) Filed: Mar. 24, 2003

Related U.S. Application Data
(60) Provisional application No. 60/392,881, filed on Jul. 1, 2002.

(51) Int. Cl. A61K 31/4439; A61K 31/047

(52) U.S. Cl. 514/343; 514/738

(57) ABSTRACT

A composition, method, and kit for the cessation of nicotine cravings. A nicotine-containing solution is provided that is applied to the oral cavity.

### CROSS-REFERENCE TO RELATED APPLICATION

This application claims priority from U.S. Provisional Application No. 60/392,881, entitled “A Composition and Method for Cessation of Nicotine Cravings,” by Patrick L. Soderlund and Raymond E. Faulk, dated Jul. 1, 2002, the contents of which are hereby incorporated in their entirety.

### FIELD OF THE INVENTION

The present invention relates to a system for the cessation of nicotine cravings. More specifically, the present invention relates to a nicotine solution, a delivery system, and methods of using both for the cessation of nicotine cravings.

### BACKGROUND OF THE INVENTION

Scientific research over the past two decades has indicated that the intake of tobacco products is unhealthy. In addition, tobacco consumption has become socially unacceptable or legally prohibited in many locations. Many people have tried and are trying to reduce or eliminate their tobacco intake.

Non-tobacco nicotine products have been introduced as tobacco substitutes and to eliminate cravings for tobacco products. Nicotine gum has been used successfully by many people as a tobacco substitute, but the gum provides a low blood nicotine level that often does not provide sufficient satisfaction to reduce cravings. Nicotine nasal sprays also reduce nicotine cravings, but cannot provide oral satisfaction. Similarly, nicotine skin patches can provide sufficient blood nicotine levels to reduce cravings. However, the patches can be unsightly and do not provide the oral satisfaction associated with tobacco intake through cigarettes or chewing tobacco.

Oral nicotine sprays also provide sufficient blood nicotine levels to reduce cravings, yet the sprays must be inhaled to be effective or require additional additives such as stimulants that can increase cost of the treatment and possibly lead to other addictions. Nicotine tablets can produce sufficient levels of nicotine in the bloodstream and provide a physical object for placement into the mouth, but the tablets dissolve during use, quickly eliminating any oral satisfaction. A nicotine solution sprayed on processed cellulosic or synthetic polymeric materials subsequently inserted into the mouth reduces cravings, but any oral satisfaction is short lived because the material dries up and causes oral irritation.

There is a need in the art for a system for the cessation of nicotine cravings that provides constant oral satisfaction and overcomes one or more of the deficiencies associated with products currently available.

### BRIEF SUMMARY OF THE INVENTION

The present invention, in one embodiment, is a composition for cessation of nicotine cravings having a nicotine solution with between about 0.1% and about 5.0% nicotine or a nicotine salt and a non-dissolvable absorbent organic carrier. Another embodiment of the present invention is a solution for oral cessation of nicotine cravings having from about 0.1% to about 5.0% nicotine or a nicotine salt and a biologically acceptable liquid carrier. Yet another embodiment is a kit for the cessation of nicotine cravings having a delivery system, a nicotine solution with from about 0.1% to about 5.0% nicotine or a nicotine salt to be dispensed by the delivery system, and an organic carrier for receiving the solution.

A further embodiment of the present invention is a treatment for the cessation of nicotine cravings. The treatment includes providing a nicotine solution having from about 0.1% to about 5.0% nicotine or a nicotine salt, applying the solution to an organic carrier, and placing the treated organic carrier in the oral cavity.

The present invention also provides methods to make compositions for the cessation of nicotine cravings. The methods include combining water, nicotine or a nicotine salt in an amount being from about 0.1% to about 5.0% of the total composition, and a sugar alcohol in an amount being from about 0.1% to about 80% of the total composition. One method of preparing compositions of the invention involve providing alcohol in an amount being from about 0.1% to about 70% of the total composition, adding nicotine or a nicotine salt in an amount being from about 0.1% to about 5.0% of the total composition, and adding a sugar alcohol in an amount being from about 0.1% to about 80% of the total composition.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

### DETAILED DESCRIPTION

The methods and compositions of the present invention are a substitute for smoking tobacco products (e.g., cigarettes, cigars, or pipe tobacco) or chewing tobacco products. To reduce cravings for such products, the nicotine containing solution of the present invention can be administered by application to the oral cavity. The nicotine in the solution is absorbed through the lining of the oral cavity, providing increased levels of nicotine in the bloodstream that lead to the reduction or elimination of cravings for tobacco products that are considered addictive. Thus, the present invention reduces or eliminates an individual's physical and/or mental dependence on tobacco products by providing a nicotine containing solution that reduces the cravings associated with tobacco products as well as providing oral satisfaction.

In one aspect, the present invention provides a nicotine solution in combination with a liquid carrier. For example, the nicotine solution can be a spray that is dispensed into the oral cavity. Once in the oral cavity, the nicotine is absorbed into the bloodstream through the mucosal tissues. Advantageously, the nicotine in the solution is absorbed through oral tissues such as the sublingual or buccal tissues. The nicotine solution provides an oral satisfaction that mimics the pleasure experienced when tobacco products are consumed. The concentration of nicotine in the solution is in an amount ranging from about 0.1% to about 5.0% by weight. The nicotine in the solution can exist as a free base or alternatively, as a free base salt. Suitable free base salts include nicotine salicylate, nicotine sulfate, nicotine malate, and nicotine citrate.

The liquid carrier can be any biologically acceptable carrier. Biologically acceptable carriers are recognized in the art and are intended to include sterile or aqueous solutions, water, salt solutions such as saline, pharmaceutically acceptable organic solvent(s) and their mixtures, pharmaceutically acceptable oils or fats, and mixtures of any and all of the above. In one embodiment, the carrier is water. Water or the biological carrier can make up from about 1.0% to about 99.9% of the nicotine solution by weight.

Alternatively, the liquid carrier can be a penetration enhancer. A penetration enhancer is a material that promotes absorption of the nicotine into the bloodstream by increasing the rate and extent of nicotine absorption. In one aspect of the invention, the penetration enhancer is an alcohol. For example, the alcohol can be ethanol. Alcohol can make up about 0.1% to about 70% by weight of the nicotine solution. In one embodiment, the alcohol also helps to solubilize the nicotine in the solution. Alternatively, the alcohol can be glycerin. Glycerin can make up about 0.1% to about 50% by weight of the nicotine solution. As an example, U.S. Pharmacopoeia (USP) grade glycerin is suitable for human consumption.

In a further embodiment, the penetration enhancer is a sugar alcohol. Examples of sugar alcohols include sorbitol, mannitol, xylitol, erythritol, inositol, perseitol, ribitol, and galactitol. The sugar alcohol in the nicotine solution of the present invention promotes the uptake of the nicotine into the bloodstream. In one aspect of the invention, the sugar alcohol in the solution also plays a role in making the solution orally satisfying in lieu of such satisfaction from tobacco products. The sugar alcohol creates a pleasant cooling effect in the oral cavity. Further, the sweet taste of the sugar alcohol improves the taste of the nicotine solution. Oral satisfaction is also enhanced by the pleasant cool and moist feeling of the liquid spray.

Alternatively, the penetration enhancer is propylene glycol. Propylene glycol can increase the rate and extent of nicotine absorption into the bloodstream.

In a further embodiment of the present invention, the nicotine solution includes a keratolytic agent. Examples of keratolytic agents include salicylic acid, salt of salicylic acid, lactic acid, and urea. The keratolytic agent can make up from about 0.001% to about 3.0% by weight of the nicotine solution. In one embodiment, the keratolytic agent improves the rate and extent of intercellular nicotine absorption into the bloodstream by causing cell swelling.

In one aspect of the present invention, the nicotine solution includes 0.1% to about 5.0% nicotine or a nicotine salt by weight with any combination of about 1.0% to about 99.9% water by weight, about 0.1% to about 70% alcohol by weight, about 0.1% to about 50% glycerin by weight, about 0.001% to about 3.0% keratolytic agent by weight, about 0.1% to about 80% sugar alcohol by weight, and/or about 0.1% to about 50% propylene glycol by weight.

In another embodiment, the solution is produced by adding a free base of nicotine or a nicotine salt to water such that the solution consists of anywhere from about 0.1% to about 5.0% nicotine or a nicotine salt by weight and from about 1.0% to about 99.9% water by weight. The solution of this embodiment may also include from about 0.001% to about 3.0% keratolytic agent by weight.

Alternatively, the solution is made by adding nicotine or a nicotine salt to alcohol in a solution in which the alcohol makes up about 0.1% to about 70% by weight. Subsequently, any combination of the following materials can be added in any order: 1.0% to about 99.9% water by weight, about 0.1% to about 50% glycerin by weight, about 0.001% to about 3.0% keratolytic agent by weight, about 0.1% to about 80% sugar alcohol by weight, and/or about 0.1% to about 50% propylene glycol by weight.

In another alternative, the nicotine solution is produced by adding nicotine or a nicotine salt in an amount being from about 0.1% to about 5.0% of the total composition to glycerin in a solution in which the glycerin makes up about 0.1% to about 50% of the total composition by weight and any combination of the following materials can be added in any order: about 0.1% to about 70% alcohol by weight, about 1.0% to about 99.9% water by weight, about 0.001% to about 3.0% keratolytic agent by weight, about 0.1% to about 80% sugar alcohol by weight, and/or about 0.1% to about 50% propylene glycol by weight.

In another alternative, the nicotine solution is produced by adding nicotine or a nicotine salt in an amount being from about 0.1% to about 5.0% of the total composition to a keratolytic agent in a solution in which the keratolytic agent makes up about 0.001% to about 3.0% of the total composition by weight and any combination of the following materials can be added in any order: about 0.1% to about 70% alcohol by weight, about 1.0% to about 99.9% water by weight, about 0.1% to about 50% glycerin by weight, about 0.1% to about 80% sugar alcohol by weight, and/or about 0.1% to about 50% propylene glycol by weight.

In a further alternative, the nicotine solution is produced by adding nicotine or a nicotine salt in an amount being from about 0.1% to about 5.0% of the total composition to a sugar alcohol in a solution in which the sugar alcohol makes up about 0.1% to about 80% of the total composition by weight and any combination of the following materials can be added in any order: about 0.1% to about 70% alcohol by weight, about 1.0% to about 99.9% water by weight, about 0.1% to about 50% glycerin by weight, about 0.001% to about 3.0% keratolytic agent by weight, and/or about 0.1% to about 50% propylene glycol by weight.

Alternatively, the nicotine solution is produced by adding nicotine or a nicotine salt in an amount being from about 0.1% to about 5.0% of the total composition to propylene glycol in a solution in which the propylene glycol makes up about 0.1% to about 50% of the total composition by weight and any combination of the following materials can be added in any order: about 0.1% to about 70% alcohol by weight, about 1.0% to about 99.9% water by weight, about 0.1% to about 50% glycerin by weight, about 0.001% to about 3.0% keratolytic agent by weight, and/or about 0.1% to about 80% sugar alcohol by weight.

According to one embodiment, a sweetening agent is added to provide oral satisfaction. The sweetening agent may be sucrolose in an amount being from about 0.001% to about 1.0% of the total composition by weight. Alternatively, the sweetening agent may be any known sweetening agent.

Flavoring can be added to provide additional oral satisfaction, according to one embodiment. The flavoring adds a pleasant taste that the individual can enjoy. Alcohol in the solution of the present invention dissolves the flavor additive into the nicotine solution. The flavor additive can be peppermint. Other examples include menthol, spearmint, menthol-peppermint, wintergreen, or other known flavors. Alternatively, the flavoring may be introduced via flavored alcohol. For example, the alcohol in the nicotine solution can be a flavored ethanol designated SDA-38B. A federal license is required to purchase SDA-38B, and it comes in various flavors, including peppermint (SDA38B-17), wintergreen (SDA-38B-15), menthol (SDA-38B-14), menthol-peppermint (SDA-38B-43), and spearmint (SDA-38B-20).

According to one aspect of the present invention, a dye is added to the nicotine solution to provide visual attractiveness. The dye can be yellow. Alternatively, the dye is blue, red, orange, aqua, purple, brown, or green. For example, the colors used can be yellow no. 5, yellow no. 6, blue no. 1, and red no. 40, where the designations are the Food, Drug, and Cosmetics designations provided by the U.S. government. From these colors, orange is produced by adding five parts yellow dye to one part red. Aqua is created by mixing five parts blue dye with one part green, while purple requires four parts red and one part blue. Brown is made with four parts yellow dye, three parts red dye, and two parts green dye, while green is made with equal parts yellow and blue dyes.

In certain embodiments, the nicotine solution can contain a stimulant. Such stimulants include caffeine, scopolamine, or any other alkaloid or stimulant. In other embodiments, the nicotine solution contains a sequestering agent. Such sequestering agents include EDTA and other similar agents.

In other certain embodiments, the nicotine solution does not contain a stimulant. Such stimulants include caffeine, scopolamine, or any other alkaloid or stimulant. In other embodiments, the nicotine solution does not contain a sequestering agent. Such sequestering agents include EDTA and other similar agents.

In one embodiment, one dose of the nicotine solution delivers about one milligram of nicotine when it is sprayed into the mouth. The dosage provides a sufficient level of nicotine in the individual's bloodstream to provide satisfaction while avoiding the levels of nicotine intake generally associated with use of tobacco products. In one aspect of the present invention, five or six doses (five or six milligrams of nicotine) per day are sufficient to reduce or eliminate nicotine cravings. This can constitute a significant reduction in an individual's nicotine intake. For example, an individual smoking two packs of cigarettes each day takes in a total of forty milligrams of nicotine (each cigarette has around 1 milligram of nicotine). By instead applying five or six doses each day of the nicotine solution of the present invention, the individual has reduced nicotine intake from 40 milligrams to five or six milligrams. Alternatively, the individual can adjust the dosage of the solution.

According to one embodiment, an aerosol sprayer can be used to dispense the nicotine solution into the mouth. Alternatively, the delivery system can be any type of sprayer or dispenser capable of dispensing a solution into the oral cavity for absorption into the blood stream through the oral tissues.

In one aspect, the present invention provides for a nicotine solution in which the active nicotine material constitutes between about 0.5% and about 1.5% of the solution by weight, or about 0.75% and about 1.25% of the solution by weight, or about 0.8% and about 1.0% of the solution by weight. According to one embodiment, variance in the concentration of nicotine by weight is accomplished by varying the amount of solution dispensed by the delivery system rather than varying the amount of active material in the solution. For example, to increase the concentration of nicotine in the solution of the present invention, the amount of liquid carrier dispensed by the delivery system is decreased. Similarly, to decrease the concentration of nicotine in the solution, the amount of liquid carrier dispensed is increased.

In another embodiment, the nicotine solution can be used in conjunction with an organic carrier. An organic carrier is any non-dissolvable, non-synthetic, biologically acceptable carrier other than processed cellulose that is produced by an organism and capable of absorbing the nicotine solution. Examples of organic carriers include cotton, natural sponge, and unprocessed plant leaves. Processed cellulosic carriers such as paper carriers irritate the oral tissues and damage the lining of the tissues as they dry. A carrier of the present invention, on the other hand, does not irritate or damage oral tissues. In certain embodiments, the carrier is not a synthetic polymer. Such synthetic polymers that are not included in this invention include olefinic polymers such as polyethylene or polypropylene, or polyolefins or polyolefindienes such as polybutadiene, poly-1-butene, polyisobutylene, polyisoprene, poly-40-methyl pentene, or combinations thereof.

The nicotine solution according to one embodiment of the present invention is sprayed onto a piece of organic material that is placed into the mouth. The nicotine solution is sprayed onto the organic carrier in a dosage that delivers about one milligram of nicotine over a period of time between about ten minutes and about ninety minutes, between about twenty minutes and about sixty minutes, or between about thirty and about forty minutes. The organic carrier is generally placed in the buccal region between the cheek and gums. Alternatively, it can be placed in the sublingual or mucosal areas of the oral cavity. The solution on the organic carrier is absorbed through the oral tissues and into the bloodstream, providing a level of nicotine that reduces cravings for tobacco products in the individual. In a different aspect of the present invention, the nicotine solution can be applied to the organic carrier by allowing the organic carrier to soak in the solution or any other method allowing for the organic carrier to absorb the solution.

Although the present invention has been described with reference to preferred embodiments, persons skilled in the art will recognize that changes may be made in form and detail without departing from the spirit and scope of the invention.

The following examples are presented by way of demonstration, and not limitation, of the invention.

### EXAMPLE 1

One embodiment of the solution of the present invention was prepared by mixing together the following components to make 100 grams of the invention:

Peppermint Menthol-Flavored Ethanol (SDA-38B-43) 40 grams

Nicotine Salicylate 1,664.32 milligrams

Propylene Glycol (USP grade) 15 grams

Glycerine (99.7% USP grade) 15 grams

Water 23.33568 grams

Sorbitol (70% USP grade) 5 grams

Dye (trace amount)

To a solution of 40 grams of ethanol was added 1,664.32 milligrams of nicotine salicylate and stirred until dissolved. To this solution was added 15 grams of propylene glycol, followed by 15 grams of glycerine, 23.33568 grams of water, and 5 grams of sorbitol. Finally, a trace of dye was added.

What is claimed is:

1. A composition for oral cessation of nicotine cravings consisting essentially of:
(a) from about 0.1% to about 5.0% nicotine or a nicotine salt; and
(b) a biologically acceptable liquid carrier.

2. The composition of claim 1 wherein the solution applied provides about one milligram of nicotine.

3. The composition of claim 1 wherein the solution does not include a stimulant.

4. The composition of claim 1 wherein the solution does not include a sequestering agent.

5. The composition of claim 1 further comprising from about 0.1% to about 50% propylene glycol.

6. The composition of claim 1 further comprising from about 0.1% to about 50% glycerin.

7. The composition of claim 1 further comprising from about 1% to about 99.9% water.

8. The composition of claim 1 further comprising from about 0.1% to about 80% sugar alcohol.

9. The composition of claim 8 wherein the sugar alcohol is selected from the group consisting of sorbitol, mannitol, xylitol, erythritol, inositol, perseitol, ribitol, and galactitol.

10. The composition of claim 1 further comprising from about about 0.001% to about 3.0% keratolytic agent.

11. The composition of claim 1 further comprising from about 0.1% to about 70% alcohol.

12. The composition of claim 1 further comprising a sweetening agent.

13. The composition of claim 12 wherein the sweetening agent is from about 0.001% to about 1.0% sucrolose.

14. The composition of claim 1 further comprising a non-dissolvable absorbent organic carrier with the proviso that the organic carrier is not a synthetic polymer or a processed cellulosic material.

15. The composition of claim 14 wherein the organic carrier is cotton.

16. A method of applying a solution for cessation of nicotine cravings comprising:
(a) providing a nicotine solution comprising from about 0.1% to about 5.0% nicotine or a nicotine salt; and
(b) placing the treated organic carrier in the oral cavity.

17. The method of claim 16 further comprising applying the solution to a non-dissolvable absorbent organic carrier with the proviso that the organic carrier is not a synthetic polymer or a processed cellulosic material.

18. The method of claim 17 wherein the placing the organic carrier in the oral cavity further comprises placing the organic carrier in the buccal region of the oral cavity.

19. The method of claim 17 wherein the placing of the organic carrier in the oral cavity further comprises placing the organic carrier in the sublinqual region of the oral cavity.

20. A kit for cessation of nicotine cravings comprising:
(a) a delivery system; and
(b) a nicotine solution comprising from about 0.1% to about 5.0% nicotine or a nicotine salt to be dispensed by the delivery system.
